(19) United States
(12) Patent Application Publication
(10) Pub. No.: US 2025/0174319 A1
(43) Pub. Date: May 29, 2025

(54) PATIENT DATA TRANSMISSION SYSTEM AND METHOD

(71) Applicant: Jong Min LEE, Gangwon-do (KR)

(72) Inventor: Jong Min LEE, Gangwon-do (KR)

(21) Appl. No.: 18/520,588

(22) Filed: Nov. 28, 2023

(30) Foreign Application Priority Data
Nov. 28, 2022 (KR) 10-2022-0162018

(51) Int. Cl. G16H 10/60; H04W 12/02

(52) U.S. Cl. CPC G16H 10/60; H04W 12/02

(57) ABSTRACT

Proposed are a patient data transmission system and method that provide a short-range communication method for de-identifying a small amount of patient data of a medical apparatus and a medical device and transmitting the de-identified data to a patient smartphone. Information transmission status may be monitored through the synchronization server, and an actual patient data value may be transmitted through video media. In the case of a device having a unique identifier, a unique value of the device is used to encrypt patient data when a QR code is created. In addition, the patient data transmission system and method may be used in transmitting a small amount of medical data, such as form papers or result values, rather than transmitting a large amount of information, and may be used in a place in which reliability of transmission is ensured because a file transmitter and receiver are in the same space.

### CROSS REFERENCE TO RELATED APPLICATION

The present application claims priority to Korean Patent Application 10-2022-0162018, filed Nov. 28, 2022, the entire contents of which is incorporated herein for all purposes by this reference.

### BACKGROUND OF THE INVENTION

### Field of the Invention

The present disclosure relates to a patient data transmission system and method. In particular, the present disclosure relates to a patient examination data transmission protocol and system using a camera and digital codes such as a QR code and a barcode.

### Description of the Related Art

Unless otherwise indicated herein, contents set forth in this section are neither the related art to the claims of the present application, nor construed as the related art despite the inclusion in this section.

Recently, medical institutions have established medical information systems to computerize all processes and records, such as a patient's reception, medical examination, prescription, treatment, hospitalization and discharge, and examination results, by using a digital technology. Specifically, the medical information systems include a medical imaging system, such as a picture archiving and communications system (PACS), for transmitting images taken with X-rays, CT, or MRI, and a hospital Information system (HIS) that integrates all of these into one.

Despite the fact that the establishment of the medical information systems is advantageous for improving public health, the medical information systems pose a high risk in terms of personal information protection. This is because medical information containing patient information data is expert and public-interest information related to an individual patient's body and is information requiring special protection among various types of personal information. Such digital data of patient information is easy to copy, so there is a possibility that personal privacy might be invaded if personal information is leaked or inaccurate information is produced. Therefore, there is a need for an effective technology for protecting the personal information, such as the patient information data, and preventing invasion of privacy.

In addition, a patient information transmission system related thereto does not have an open wired and wireless convergence coordinator platforms required by the Internet of things (IoT) system. Thus, due to the use of multi-services in the ISM band, such as wireless Internet, Bluetooth, and Zigbee, communication interference phenomena are getting worse day by day in hospital environments, making real-time information changes impossible, causing low transmission reliability, and making the realization of low-power communication impossible.

The foregoing is intended merely to aid in the understanding of the background of the present disclosure, and is not intended to mean that the present disclosure falls within the purview of the related art that is already known to those skilled in the art.

### DOCUMENTS OF RELATED ART

(Patent Document 1) Korean Patent No. 10-1391773 (28 Apr. 2014); and
(Patent Document 2) Korean Patent No. 10-1068687 (22 Sep. 2011).

### SUMMARY OF THE INVENTION

A patient data transmission system and method according to embodiments provide a short-range communication method for de-identifying a small amount of patient data of the medical apparatus and a medical device and transmitting the de-identified data to a patient smartphone.

In the embodiments, information transmission status may be monitored through the synchronization server, and an actual patient data value may be transmitted through video media rather than a network.

In addition, in the embodiments, in the case of a device, such as a smartphone, having a unique identifier, a unique value of the device is used to encrypt patient data when a QR code is created.

In addition, the patient data transmission system and method according to the embodiments may be used in transmitting a small amount of medical data, such as form papers or result values, rather than transmitting a large amount of information. The patient data transmission system and method may be used in a place in which reliability of transmission is ensured because a file transmitter and receiver are in the same space.

The patient data transmission system according to the embodiment includes: a medical apparatus configured to create patient examination information including a patient's reception, medical examination, prescription, treatment, hospitalization and discharge, and an examination result, and create synchronization information allowing the patient examination information to be checked; a smart terminal configured to receive the patient examination information created by the medical apparatus; and a synchronization server configured to receive, from the medical apparatus, the synchronization information allowing the patient examination information to be checked on the smart terminal, and transmit the synchronization information to the smart terminal when authentication of the smart terminal is completed.

In the embodiment, the smart terminal is configured to receive, after authentication with the synchronization server is completed, a synchronization value for checking the patient examination information from the synchronization server and receive patient information directly from the medical apparatus to output the patient information.

In the embodiment, the synchronization server may include: a communication module configured to receive the patient examination information obtained by performing de-identification from the medical apparatus, and transmit the synchronization information of the received patient examination information to the smart terminal; a synchronization module configured to determine whether the patient examination information stored in the medical apparatus and the patient examination information stored in the smart terminal are the same; and a creation module configured to create the synchronization value for checking the patient examination information and transmit the synchronization value to the smart terminal when the synchronization module determines that the patient examination information stored in the medical apparatus and the patient examination information stored in the smart terminal are the same.

In the embodiment, the medical apparatus may include: a patient examination information collection module configured to store the patient examination information including the reception, the medical examination, the prescription, the treatment, hospitalization and discharge results, and an X-ray, CT, or MRI imaging result of the patient; a de-identification module configured to de-identify the collected patient examination information; an authentication module configured to detect the smart terminal that is capable of short-range communication and device-to-device communication with the medical apparatus, and authenticate the detected smart terminal; and an examination information transmission module configured to transmit the patient examination information to the authenticated smart terminal.

In the embodiment, the smart terminal may include: an authentication code creation module configured to create a user authentication code including a QR code or a barcode for receiving patient examination result information and the synchronization information; an encryption module configured to encrypt the created user authentication code using a unique identifier of the smart terminal; a synchronization information collection module configured to receive the synchronization information including the synchronization value from the synchronization server when the synchronization server completes authentication of the smart terminal successfully with the created user authentication code; and an examination information output module configured to receive the patient examination information directly from the medical apparatus and output the patient examination information when it is determined that the smart terminal is positioned within a particular distance of the medical apparatus.

The patient data transmission system and method described above improves the security of patient data by preventing data exposure to hacking situations through the network. In addition, the synchronization server provided in the embodiment stores only a profile for transmission details, so the security of content included in the actual transmission file can be improved.

It should be understood that the effects are not limited to those described above, but include all effects that can be inferred from the configurations of the disclosure in the detailed description of the present disclosure or the claims.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a diagram illustrating the configuration of a patient data transmission system according to an embodiment;

FIG. 2 is a diagram illustrating the data processing configuration of a medical apparatus according to the embodiment;

FIG. 3 is a diagram illustrating the data processing configuration of a synchronization server according to the embodiment;

FIG. 4 is a diagram illustrating the data processing configuration of a smart terminal according to the embodiment;

FIG. 5 is a diagram illustrating the signal flow of a patient data transmission system according to an embodiment;

FIG. 6 is a diagram illustrating the data processing flow of a medical apparatus according to the embodiment;

FIG. 7 is a diagram illustrating the data processing flow of a synchronization server according to the embodiment; and

FIG. 8 is a diagram illustrating the data processing configuration of a smart terminal according to the embodiment.

### DETAILED DESCRIPTION OF THE INVENTION

Advantages and features of the present disclosure, and methods to achieve them will be apparent from the following embodiments that will be described in detail with reference to the accompanying drawings. It should be understood that the present disclosure is not limited to the following embodiments and may be embodied in different ways, and that the embodiments are provided to only complete the disclosure and to allow those skilled in the art to fully understand the category of the disclosure. The disclosure is defined by the category of the claims. Throughout the specification, the same reference numerals refer to the same elements.

In describing the embodiments of the present disclosure, when if it is decided that a detailed description of a well-known function or configuration makes the gist of the present disclosure unclear, the detailed description will be omitted. Further, the terms described below are defined in consideration of the functions in the embodiments of the present disclosure, and may vary depending on the intention of the user, the operator, or the custom. Therefore, the definitions should be based on the contents throughout this specification.

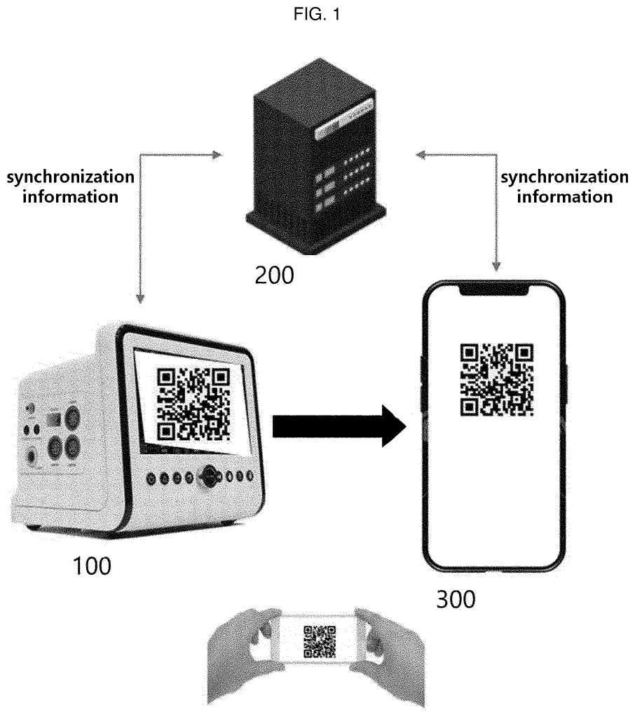

FIG. 1 is a diagram illustrating the configuration of a patient data transmission system according to an embodiment.

Referring to FIG. 1, the patient data transmission system according to the embodiment may include a medical apparatus 100, a synchronization server 200, and a smart terminal 300. The medical apparatus 100 creates patient examination information and synchronization information. The patient examination information includes a patient's reception, medical examination, prescription, treatment, hospitalization and discharge, and an examination result. The synchronization information allows the patient examination information to be checked.

The smart terminal 300 receives the patient examination information created by the medical apparatus. The synchronization server 200 receives, from the medical apparatus 100, the synchronization information that allows the patient examination information received by the smart terminal to be checked. In the embodiment, when authentication of the smart terminal is completed, the synchronization server 200 transmits the synchronization information to the smart terminal. In the embodiment, the smart terminal 300 uses the patient personal information to create a digital code, such as a QR code or a barcode, as an authentication code such that the patient and the smart terminal owned by the patient are authenticated in the synchronization server and the medical apparatus.

In the embodiment, after authentication with the synchronization server is completed, the smart terminal 300 receives, from the synchronization server, a synchronization value for checking the patient examination information. The synchronization value may include a key value for streaming and outputting the patient information transmitted to the smart terminal. In the embodiment, when the smart terminal 300 receives the synchronization value from the synchronization server, the synchronization value is automatically input in outputting the patient information is output.

In the embodiment, the data transmitted from the medical apparatus to the smart terminal may be monitored through the synchronization server. In addition, the patient data transmission system according to the embodiment may provide both a method of transmitting patient data to the smart terminal by using the synchronization server and a method of transmitting patient data to the smart terminal without the synchronization server.

The patient data transmission system and method according to the embodiments provide a short-range communication method for de-identifying a small amount of patient data of the medical apparatus and a medical device and transmitting the de-identified data to a patient smartphone. In the embodiments, information transmission status may be monitored through the synchronization server, and an actual patient data value may be transmitted through video media rather than a network. In the embodiments, in the case of a device, such as a smartphone, with a unique identifier, a unique value of the device is used to encrypt the patient data when an authentication code, such as a QR code, is created. In addition, the patient data transmission system and method according to the embodiments may be used in transmitting a small amount of medical data, such as form papers or result values, rather than transmitting a large amount of information. The patient data transmission system and method may be used in a place in which reliability of transmission is ensured because a file transmitter and receiver are in the same space.

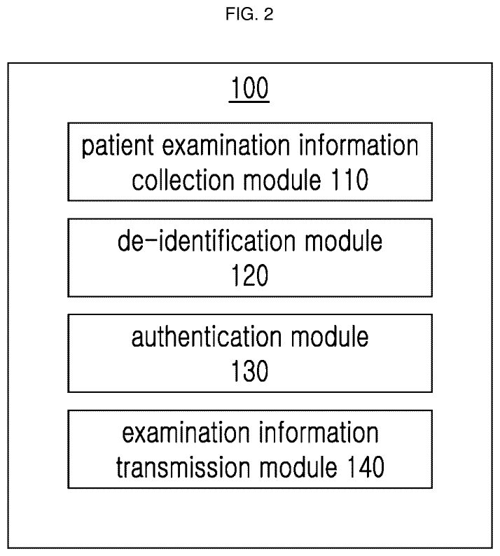

FIG. 2 is a diagram illustrating the data processing configuration of the medical apparatus according to the embodiment.

Referring to FIG. 2, the medical apparatus 100 according to the embodiment may include a patient examination information collection module 110, a de-identification module 120, an authentication module 130, and an examination information transmission module 140. The term “module” used herein should be interpreted to include software, hardware, or a combination thereof, depending on the context in which the term is used. For example, software may be machine language, firmware, embedded code, and application software. As another example, hardware may be a circuit, a processor, a computer, an integrated circuit, an integrated circuit core, a sensor, a micro-electro-mechanical system (MEMS), a passive device, or a combination thereof.

The patient examination information collection module 110 stores, for each patient, patient examination information including the patient's reception, medical examination, prescription, treatment, hospitalization and discharge results, and an X-ray, CT, or MRI imaging result.

The de-identification module 120 de-identifies the collected patient examination information. De-identification provide in the embodiment is a data processing process used to prevent someone's identify from being revealed. For example, regarding data generated during human experiments, the collected data may be de-identified to protect the privacy of research participants. When applied to metadata or general data about identification, this process is also called data anonymization. In the embodiment, the de-identification module 120 may de-identify examination information of a patient by deleting or masking personal identifiers, such as a person's name, and by omitting or generalizing quasi-identifiers, such as date of birth. In addition, a series of measures may be taken to make it impossible to identify a specific individual by deleting or replacing some or all of the personal information or processing the personal information so that the personal information cannot be easily combined with other information. Through this, the possibility of personal information infringement is minimized and there is no information leakage when examination information of a patient is transmitted and received or used as big data.

In the embodiment, the de-identification module 120 may perform de-identification of the personal information by using a camera sensor or de-identification operations including heuristic pseudonymization and encryption swapping. In the embodiment, heuristic pseudonymization hides detailed personal information by replacing values corresponding to identifiers with several set rules or by processing the values according to human judgment. In the embodiment, a file name, a serial number, and image provider personal information of an encrypted image may be hidden. Encryption is to replace personal information through encryption by applying an algorithm with particular rules when information is processed. In the embodiment, the resident registration number, passport number, medical insurance number, foreign registration number, user ID, credit card number, or biometric information of a user who has provided an image may be encrypted. In addition, the de-identification module 120 may de-identify information on a user who has provided patient information including an image, through the swapping. The swapping according to the embodiment is exchanging records in an existing database for predetermined external variable (item) values in association therewith. In the embodiment, personal information, such as a user ID, a medical care institution number, an institution number, age, sex, physical information (height, blood type, etc.), income, a mobile phone number, or an address, may be exchanged for the predetermined external variable (item) values in association therewith.

The authentication module 130 detects a smart terminal that is positioned within a particular distance of the medical apparatus and is thus capable of short-range communication, and authenticates the detected smart terminal. In the embodiment, authentication of the user of the smart terminal or device authentication may be performed. In the embodiment, the authentication module 130 receives, from the smart terminal, authentication code information created using the personal information, and compares the patient's personal information included in the received authentication code to the stored patient information, and completes the authentication of the smart terminal according to a result of comparison. In addition, the medical apparatus recognizes an authentication code output on a display window of the smart terminal, and compares the patient information included in the recognized authentication code with the patient information stored in the medical apparatus, and determines whether the smart terminal is authenticated according to a result of comparison. In the embodiment, when the patient's personal information included in the authentication code and the patient's personal information stored in the medical apparatus are the same, authentication is successfully completed.

The examination information transmission module 140 transmits the patient examination information to the authenticated smart terminal. In the embodiment, the examination information transmission module 140 transmits the patient examination information to the smart terminal using a device-to-device (D2D) communication method and short-range communication, such as near-field communication (NFC), Zigbee, or Zing technology, which enables data transmission at ultra-close distances. D2D communication is a technology that enables direct communication between terminals without passing through a base station or an AP, thus providing a service to a user while reducing the load on the switching network. In the embodiment, the examination information transmission module 140 may directly transmit, to the authenticated smart terminal, the examination information of the patient who owns the smart terminal. In the embodiment, the examination information transmission module 140 may directly transmit, to the authenticated smart terminal, the examination information of the patient who owns the smart terminal. In the embodiment, the smart terminal, which is a device for playing patient examination information back, connects to the medical apparatus, which is a medium in which the patient examination information is stored, using a short-range wireless communication or direct transmission method, and the smart terminal begins to receive streaming of the patient information from the medical apparatus. Afterward, in the embodiment, the strength of a signal transmitted by the medical apparatus or the error rate of data transmitted by the medical apparatus is measured a particular number of times for a reference period of time, starting from the point in time when the smart terminal receives the streaming of the patient examination information or from a reference point in time. When the signal strength is equal to or greater than a first reference value or when the error rate of data transmitted by the medium is equal to or less than a second reference value, the smart terminal receives content without exchanging ACK messages with the medical apparatus.

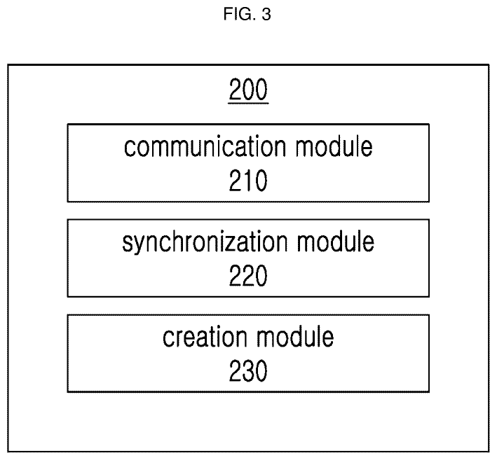

FIG. 3 is a diagram illustrating the data processing configuration of the synchronization server according to the embodiment.

Referring to FIG. 3, the synchronization server 200 according to the embodiment may include a communication module 210, a synchronization module 220, and a creation module 230. The communication module 210 receives the de-identified patient examination information from the medical apparatus, and transmits the synchronization information of the received patient examination information to the authenticated smart terminal. The synchronization module 220 determines whether the patient examination information stored in the medical apparatus and the patient examination information stored in the authenticated smart terminal are the same.

When the synchronization module 220 determines that the patient examination information stored in the medical apparatus and the patient examination information stored in the smart terminal are the same, the creation module 230 creates a synchronization value for checking the patient examination information and transmits the synchronization value to the smart terminal. In the embodiment, the synchronization value may be created through the synchronization information received from the medical apparatus.

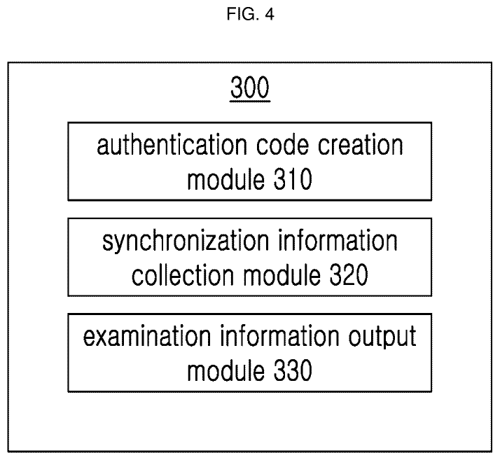

FIG. 4 is a diagram illustrating the data processing configuration of the smart terminal according to the embodiment.

Referring to FIG. 4, the smart terminal 300 according to the embodiment may include an authentication code creation module 310, a synchronization information collection module 320, and an examination information output module 330. The authentication code creation module 310 creates a user authentication code for receiving patient examination result information and synchronization information including a synchronization value. Examples of the user authentication code include a QR code and a barcode. In the embodiment, the user authentication code may be created using the user's personal information, such as the patient's resident registration number, date of birth, ID, or examination date and time. The encryption module 320 encrypts the created user authentication code using a unique identifier of the smart terminal.

The synchronization information collection module 320 receives the synchronization information from the synchronization server when the authentication of the smart terminal is successfully completed with the created user authentication code. In the embodiment, the synchronization information includes data, such as a data output secret code or a final data update point in time, for determining whether the patient information to be output from the smart terminal is the same as the patient information output to the medical apparatus. In the embodiment, the synchronization information may include a secret key value obtained by encrypting the final update point in time, the patient's resident registration number, and the patient number.

When it is determined that the smart terminal is positioned within a particular distance of the medical apparatus, the examination information output module 330 receives the patient examination information from the medical apparatus through short-range communication or device-to-device communication and outputs the received patient information. In the embodiment, the examination information output module 330 may output the patient examination information through video streaming using the short-range wireless communication method and the device-to-device communication method. In the embodiment, when the smart terminal receives the synchronization information from the synchronization server, the synchronization value is automatically input to the received patient examination information. When the input synchronization value matches the synchronization value set for the patient examination information, the smart terminal may output the patient information.

Hereinafter, a patient data transmission method will be described in order. The operation (function) of the patient data transmission method according to an embodiment is fundamentally the same as the function of the patient data transmission system, so a description the same as that of FIGS. 1 to 4 will be omitted.

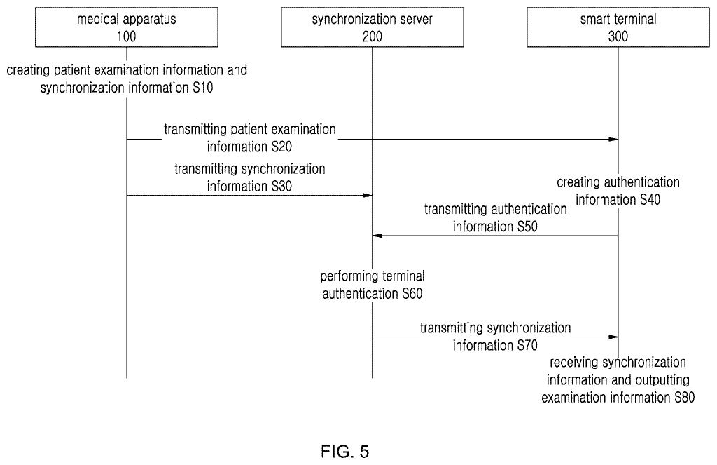

FIG. 5 is a diagram illustrating the signal flow of a patient data transmission system according to an embodiment.

Referring to FIG. 5, in step S10, the medical apparatus creates patient examination information including a patient's reception, medical examination, prescription, treatment, hospitalization and discharge, and an examination result, and creates synchronization information that allows the patient examination information to be checked. In step S20, the patient examination information is transmitted to the smart terminal. In step S30, the medical apparatus transmits the synchronization information to the synchronization server. In step S40, the smart terminal uses the patient's personal information to create authentication information. In step S50, the authentication information is transmitted to the synchronization server or the authentication information is input to the synchronization server. In step S60, the recognized or received authentication information is compared to stored data to perform terminal authentication. When the smart terminal is successfully authenticated, the synchronization information including a synchronization value is transmitted to the smart terminal in step S70. In step S80, the smart terminal receives the synchronization information and then outputs the patient examination information.

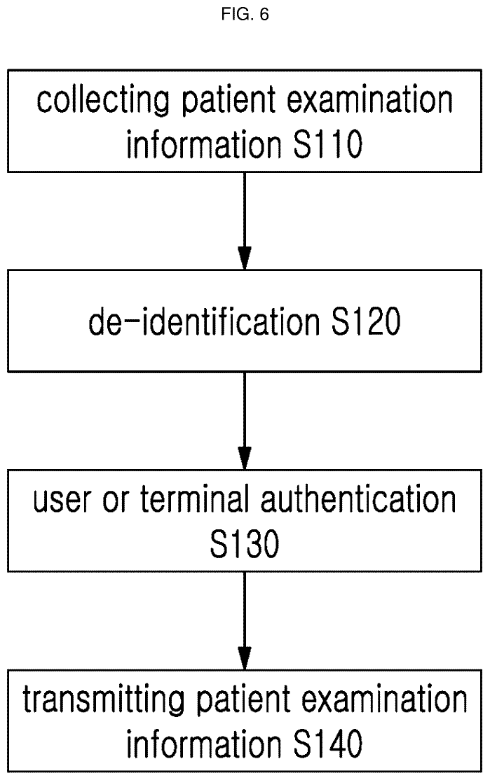

FIG. 6 is a diagram illustrating the data processing flow of the medical apparatus according to the embodiment.

Referring to FIG. 6, in step S110, the patient examination information collection module store, for each patient, patient examination information including the patient's reception, medical examination, prescription, treatment, hospitalization and discharge results, and an X-ray, CT, or MRI imaging result.

In step S120, the de-identification module de-identifies the collected patient examination information. De-identification provide in the embodiment is a data processing process used to prevent someone's identify from being revealed. For example, regarding data generated during human experiments, the collected data may be de-identified to protect the privacy of research participants. When applied to metadata or general data about identification, this process is also called data anonymization. In the embodiment, in step S120, examination information of a patient may be de-identified by deleting or masking personal identifiers, such as a person's name, and by omitting or generalizing quasi-identifiers, such as date of birth. In addition, a series of measures may be taken to make it impossible to identify a specific individual by deleting or replacing some or all of the personal information or processing the personal information so that the personal information cannot be easily combined with other information. Through this, the possibility of personal information infringement is minimized and there is no information leakage when examination information of a patient is transmitted and received or used as big data. In the embodiment, de-identification of the personal information may be performed using a camera sensor or de-identification operations including heuristic pseudonymization and encryption swapping. In the embodiment, heuristic pseudonymization hides detailed personal information by replacing values corresponding to identifiers with several set rules or by processing the values according to human judgment. In the embodiment, a file name, a serial number, and image provider personal information of an encrypted image may be hidden. Encryption is to replace personal information through encryption by applying an algorithm with particular rules when information is processed. In the embodiment, the resident registration number, passport number, medical insurance number, foreign registration number, user ID, credit card number, or biometric information of a user who has provided an image may be encrypted. In addition, the de-identification module 120 may de-identify information on a user who has provided patient information including an image, through the swapping. The swapping according to the embodiment is exchanging records in an existing database for predetermined external variable (item) values in association therewith. In the embodiment, personal information, such as a user ID, a medical care institution number, an institution number, age, sex, physical information (height, blood type, etc.), income, a mobile phone number, or an address, may be exchanged for the predetermined external variable (item) values in association therewith.

In step S130, the authentication module detects a smart terminal that is positioned within a particular distance of the medical apparatus and is thus capable of short-range communication, and authenticates the detected smart terminal. In the embodiment, in step S130, authentication code information created using the personal information is received from the smart terminal, and the patient's personal information included in the received authentication code is compared to the stored patient information, and the authentication of the smart terminal is completed according to a result of comparison. In addition, the medical apparatus recognizes an authentication code output on a display window of the smart terminal, and compares the patient information included in the recognized authentication code with the patient information stored in the medical apparatus, and determines whether the smart terminal is authenticated according to a result of comparison.

In step S140, the examination information transmission module transmits the patient examination information to the authenticated smart terminal. In the embodiment, the examination information transmission module 140 transmits the patient examination information to the smart terminal using a device-to-device (D2D) communication method and short-range communication, such as near-field communication (NFC), Zigbee, or Zing technology, which enables data transmission at ultra-close distances. D2D communication is a technology that enables direct communication between terminals without passing through a base station or an AP, thus providing a service to a user while reducing the load on the switching network. In the embodiment, in step S140, the examination information of the patient who owns the smart terminal may be directly transmitted to the authenticated smart terminal. In the embodiment, in step S140, the examination information of the patient who owns the smart terminal may be directly transmitted to the authenticated smart terminal. In the embodiment, the smart terminal, which is a device for playing patient examination information back, connects to the medical apparatus, which is a medium in which the patient examination information is stored, using a short-range wireless communication or direct transmission method, and the smart terminal begins to receive streaming of the patient information from the medical apparatus. Afterward, in the embodiment, the strength of a signal transmitted by the medical apparatus or the error rate of data transmitted by the medical apparatus is measured a particular number of times for a reference period of time, starting from the point in time when the smart terminal receives the streaming of the patient examination information or from a reference point in time. When the signal strength is equal to or greater than a first reference value or when the error rate of data transmitted by the medium is equal to or less than a second reference value, the smart terminal receives content without exchanging ACK messages with the medical apparatus.

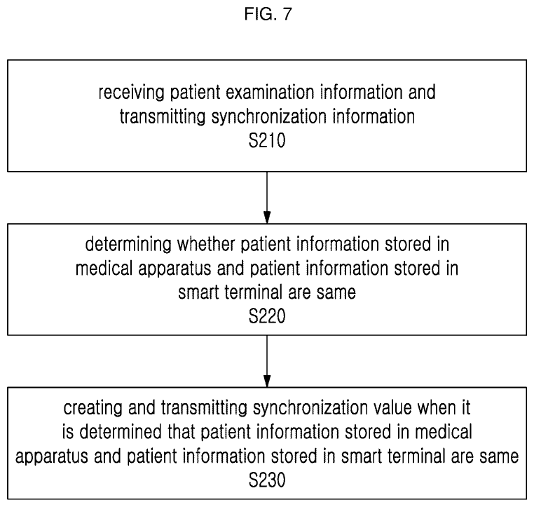

FIG. 7 is a diagram illustrating the data processing flow of the synchronization server according to the embodiment.

Referring to FIG. 7, in step S210, the communication module receives the de-identified patient examination information from the medical apparatus, and transmits the synchronization information of the received patient examination information to the authenticated smart terminal. In step S220, the synchronization module determines whether the patient examination information stored in the medical apparatus and the patient examination information stored in the authenticated smart terminal are the same.

In step S230, when it is determined that the patient examination information stored in the medical apparatus and the patient examination information stored in the smart terminal are the same, the creation module creates a synchronization value for checking the patient examination information and transmits the synchronization value to the smart terminal.

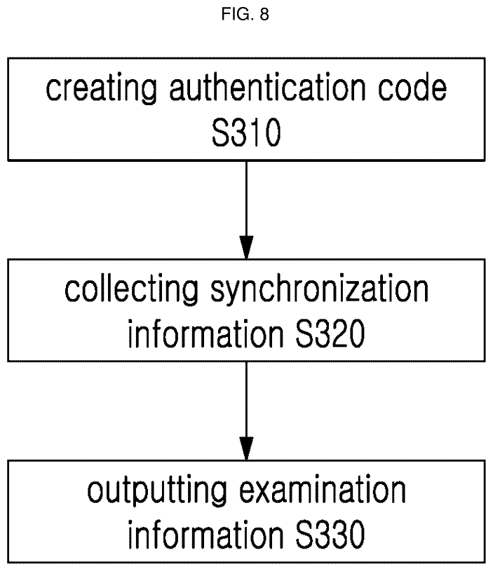

FIG. 8 is a diagram illustrating the data processing configuration of the smart terminal according to the embodiment.

Referring to FIG. 8, in step S310, the authentication code creation module 310 creates a user authentication code including a QR code or a barcode for receiving patient examination result information and synchronization information. In the embodiment, the user authentication code may be created using the user's personal information, such as the patient's resident registration number, date of birth, ID, or examination date and time. The encryption module 320 encrypts the created user authentication code using a unique identifier of the smart terminal.

In step S320, when authentication of the smart terminal is successfully completed with the created user authentication code, the synchronization information collection module 320 receives the synchronization information from the synchronization server. In the embodiment, the synchronization information includes data, such as a data output secret code or a final data update point in time, for determining whether the patient information to be output from the smart terminal is the same as the patient information output to the medical apparatus.

In step S330, when it is determined that the smart terminal is positioned within a particular distance of the medical apparatus, the examination information output module 330 receives the patient examination information from the medical apparatus through short-range communication or device-to-device communication and outputs the received patient information. In the embodiment, in step S330, patient examination information may be output through video streaming using the short-range wireless communication method and the device-to-device communication method.

The patient data transmission system and method described above improves the security of patient data by preventing data exposure to hacking situations through the network. In addition, the synchronization server provided in the embodiment stores only a profile for transmission details, so the security of content included in the actual transmission file can be improved.

Although preferred embodiments of the present disclosure have been described for illustrative purposes, those skilled in the art will appreciate that various modifications, additions, and substitutions are possible, without departing from the scope and spirit of the disclosure as disclosed in the accompanying claims.

What is claimed is:

1. A patient data transmission system, comprising:
a medical apparatus configured to create patient examination information including a patient's reception, medical examination, prescription, treatment, hospitalization and discharge, and an examination result, and create synchronization information allowing the patient examination information to be checked;
a smart terminal configured to receive the patient examination information created by the medical apparatus; and
a synchronization server configured to receive, from the medical apparatus, the synchronization information allowing the patient examination information to be checked on the smart terminal, and transmit the synchronization information to the smart terminal when authentication of the smart terminal is completed,
wherein the smart terminal is configured to receive, after authentication with the synchronization server is completed, a synchronization value for checking the patient examination information from the synchronization server and receive patient information directly from the medical apparatus to output the patient information.

2. The patient data transmission system of claim 1, wherein the synchronization server comprises:
a communication module configured to receive the patient examination information obtained by performing de-identification from the medical apparatus, and transmit the synchronization information of the received patient examination information to the smart terminal;
a synchronization module configured to determine whether the patient examination information stored in the medical apparatus and the patient examination information stored in the smart terminal are the same; and
a creation module configured to create the synchronization value for checking the patient examination information and transmit the synchronization value to the smart terminal when the synchronization module determines that the patient examination information stored in the medical apparatus and the patient examination information stored in the smart terminal are the same.

3. The patient data transmission system of claim 1, wherein the medical apparatus comprises:
a patient examination information collection module configured to store the patient examination information including the reception, the medical examination, the prescription, the treatment, hospitalization and discharge results, and an X-ray, CT, or MRI imaging result of the patient;
a de-identification module configured to de-identify the collected patient examination information;
an authentication module configured to detect the smart terminal that is capable of short-range communication and device-to-device communication with the medical apparatus, and authenticate the detected smart terminal; and
an examination information transmission module configured to transmit the patient examination information to the authenticated smart terminal.

4. The patient data transmission system of claim 1, wherein the smart terminal comprises:
an authentication code creation module configured to create a user authentication code including a QR code or a barcode for receiving patient examination result information and the synchronization information;
an encryption module configured to encrypt the created user authentication code using a unique identifier of the smart terminal;
a synchronization information collection module configured to receive the synchronization information including the synchronization value from the synchronization server when the synchronization server completes authentication of the smart terminal successfully with the created user authentication code; and
an examination information output module configured to receive the patient examination information directly from the medical apparatus and output the patient examination information when it is determined that the smart terminal is positioned within a particular distance of the medical apparatus.

5. The patient data transmission system of claim 4, wherein the examination information output module is configured to output the patient examination information through video streaming using short-range wireless communication.

6. A patient data transmission method, comprising:
(A) creating, by a medical apparatus, patient examination information including a patient's reception, medical examination, prescription, treatment, hospitalization and discharge, and an examination result, and creating synchronization information allowing the patient examination information to be checked;
(B) receiving, by a smart terminal, the patient examination information created by the medical apparatus;
(C) receiving, by a synchronization server from the medical apparatus, the synchronization information allowing the patient examination information to be checked on the smart terminal, and transmitting the synchronization information to the smart terminal when authentication of the smart terminal is completed; and
(D) receiving, by the smart terminal after authentication with the synchronization server is completed, a synchronization value for checking the patient examination information from the synchronization server, and receiving patient information directly from the medical apparatus to output the patient information.

7. The patient data transmission method of claim 6, wherein the step (C) comprises:
receiving the patient examination information obtained by performing de-identification from the medical apparatus, and transmitting the synchronization information of the received patient examination information to the smart terminal;
determining whether the patient examination information stored in the medical apparatus and the patient examination information stored in the smart terminal are the same; and
creating the synchronization value for checking the patient examination information and transmitting the synchronization value to the smart terminal when the synchronization module determines that the patient examination information stored in the medical apparatus and the patient examination information stored in the smart terminal are the same.

8. The patient data transmission method of claim 6, wherein the step (A) comprises:
a patient examination information collection module configured to store the patient examination information including the reception, the medical examination, the prescription, the treatment, hospitalization and discharge results, and an X-ray, CT, or MRI imaging result of the patient;
de-identifying the collected patient examination information;
detecting the smart terminal that is capable of short-range communication and device-to-device communication with the medical apparatus, and authenticating the detected smart terminal; and
transmitting the patient examination information to the authenticated smart terminal.

9. The patient data transmission method of claim 6, wherein the step (B) comprises:
creating a user authentication code including a QR code or a barcode for receiving patient examination result information and the synchronization information;
encrypting the created user authentication code using a unique identifier of the smart terminal;
receiving the synchronization information including the synchronization value from the synchronization server when the synchronization server completes authentication of the smart terminal successfully with the created user authentication code; and
receiving the patient examination information directly from the medical apparatus and outputting the patient examination information when it is determined that the smart terminal is positioned within a particular distance of the medical apparatus.

10. The patient data transmission method of claim 9, wherein in the receiving of the patient examination information directly from the medical apparatus and outputting of the patient examination information when it is determined that the smart terminal is positioned within the particular distance of the medical apparatus, the patient examination information is output through video streaming using short-range wireless communication.
